(19) United States
(12) Patent Application Publication
(10) Pub. No.: US 2017/0119575 A1
(43) Pub. Date: May 4, 2017

(54) METHOD AND APPARATUS UTILIZING MAGNETIC NANOPARTICLES FOR PERFORMING HYPERTHERMAL THERAPIES

(71) Applicant: The Trustees of Dartmouth College, Hanover, NH (US)

(72) Inventors: Fridon SHUBITIDZE, Hanover, NH (US); Katsiaryna KEKALO, Lebanon, NH (US); B. Stuart TREMBLY, Hanover, NH (US); Ian BAKER, Etna, NH (US)

(21) Appl. No.: 15/406,681

(22) Filed: Jan. 14, 2017

Related U.S. Application Data
(63) Continuation of application No. PCT/US2015/040302, filed on Jul. 14, 2015.
Application No. PCT/US2015/040302 is a continuation-in-part of application No. 13/717,527, filed on Dec. 17, 2012.
Application No. 13/717,527 is a continuation of application No. PCT/US2011/040722, filed on Jun. 16, 2011.
(60) Provisional application No. 62/118,538, filed on Feb. 20, 2015.
(60) Provisional application No. 62/024,296, filed on Jul. 14, 2014.
(60) Provisional application No. 61/355,407, filed on Jun. 16, 2010.

(51) Int. Cl. A61F 7/12; A61N 2/02; A61N 2/00

(52) U.S. Cl. CPC A61F 7/12; A61N 2/004; A61N 2/02; A61F 2007/126; A61F 2007/0096

(57) ABSTRACT

A system for heating tissue in a subject, has an alternating current (AC) source coupled to a coil; a magnetically permeable passive magnetic concentrator, and positioned to alter an intensity distribution of the field;. The system has a processor with a computer model of coil and concentrator, the memory with code for simulating magnetic fields and configured to generate an intensity map describing the intensity distribution of the AC magnetic field. A method of providing an alternating current (AC) magnetic field to magnetic nanoparticles includes: providing a source of alternating current coupled to a coil; positioning the coil in a vicinity of the nanoparticles; positioning a passive concentrator near the nanoparticles; and energizing the coil to generate the AC field, the field shaped by the concentrator and heats the nanoparticles. A particular embodiment simulates the magnetic field and compares the field to parameters to verify adequate heating of the nanoparticles.

### RELATED APPLICATION

The present application is a continuation of International Application No. PCT/US2015/040302, filed 14 Jul. 2015 which claims the benefit of priority to U.S. Patent Application No. 62/024,296 filed 14 Jul. 2014 and U.S. Provisional Patent Application No. 62/118,538 filed 20 Feb. 2015. The entire content of each of these applications is incorporated herein by reference.

The present application is also a continuation-in-part of U.S. patent application Ser. No. 13/717,527 filed 17 Dec. 2012 which is a continuation of International Application No. PCT/US2011/040722 filed 16 Jun. 2011 which claims the benefit of priority to U.S. Provisional Application No. 61/355,407 filed 16 Jun. 2010. The entire content of each of these applications is incorporated herein by reference.

The present application is also related to U.S. patent application Ser. No. 13/945,610, now U.S. Pat. No. 9,271,789, filed 18 Jul. 2013, which claims the benefit of priority to U.S. Provisional Application No. 61/672,991 filed 18 Jul. 2012.

### GOVERNMENT RIGHTS

This invention was made with government support under contract no. U54CA151662 awarded by the National Institutes of Health. The government has certain rights in the invention

### BACKGROUND

Magnetic nanoparticles have been used for hyperthermal therapy of particular organs and/or tumors. In performing hyperthermal therapy, the nanoparticles are applied to the organs and/or tumors, typically by injection either into the organ or tumor, or into a blood vessel feeding the organ, followed by application of AC magnetic fields. The AC magnetic fields stimulate the nanoparticles, thereby heating the nanoparticles, which in turn heat the organ or tumor.

When performing hyperthermal therapy, it is desirable to focus heat in selected tissues, tissues such as tumors or other abnormal tissues that the therapist wishes to destroy. Similarly, it is desirable to limit heating of other tissues, including nearby normal tissues so prevent undue harm to patients. It is the desire to focus heat in selected tissues that motivates use of magnetic nanoparticles because it is believed that nanoparticles offer opportunities to heat selected, deep tissues, organs, and tumors, with less surface heating and less heating of nearby normal tissues than with available alternatives such as diathermy machines.

Even though prior magnetic nanoparticle thermotherapy techniques focus deep heating in ways impossible with prior technologies, the AC magnetic fields used can still cause some undesired heating of surface tissues, and of tissues near targeted tissues, organs, and tumors. Although mammalian tissue is not ferromagnetic, it contains significant salts and water, and is electrically conductive. Some of this undesired heating is due to eddy currents induced by the AC magnetic fields in electrically conductive tissue.

In prior studies of magnetic nanoparticle heating of tissues, after infiltration of nanoparticles into selected tissues, AC magnetic fields are typically applied using a coil placed external to the body. A way of controlling what tissues are heated includes having the magnetic nanoparticles in high concentrations where thermotherapy is desired—such as in a tumor—and low concentrations or absent elsewhere.

We and our coworkers have previously disclosed magnetic nanoparticle tissue-heating systems that make use of a flexible magnetic rod that has a coil wound on one end of the rod, and a second end adapted for insertion into openings, such as the vaginal opening, or, through an endoscope, to a tumor located in the gut.

For purposes of this document, magnetic nanoparticles are particles in the size range of from ten to five thousand nanometers diameter, where a significant proportion of each particle is formed of a magnetic material such as but not limited to iron or iron oxide.

### SUMMARY

In an embodiment, a system is adapted for applying heat to a treatment location in a subject, the system including an alternating current (AC) source coupled to energize a coil configured to generate an AC magnetic field; a magnetically permeable, passive magnetic concentrator formed of a magnetically permeable material, the concentrator not positioned within the coil, the concentrator positioned to alter an intensity distribution of the AC magnetic field; and apparatus configured to administer magnetic nanoparticles to tissue to be treated of a subject. In particular embodiments, the system has a processor coupled to a memory, the memory containing a computer model of the coil and the concentrator, the memory further containing computer readable code for simulating magnetic fields and configured to generate an intensity map describing the intensity distribution of the AC magnetic field.

In another embodiment, a method of providing an alternating current (AC) magnetic field to magnetic nanoparticles and heating the magnetic nanoparticles includes: providing a source of alternating current coupled to a coil; positioning the coil (and any associated active concentrator) to provide magnetic field in a vicinity of the nanoparticles; positioning a passive concentrator near the nanoparticles to adjust the field; and energizing the coil with the alternating current to generate the AC magnetic field. A particular embodiment also includes simulating the AC magnetic field and comparing the magnetic field to parameters to verify adequate heating of the nanoparticles.

### BRIEF DESCRIPTION OF THE FIGURES

FIG. 1 is an embodiment of our previously disclosed magnetic nanoparticle tissue-heating system using a flexible magnetically-permeable rod having magnetic field concentration properties to heat fallopian tubes.

FIG. 2 illustrates placement of an endoscope tip near tissue to be treated, such that a nanoparticle injector may be passed through a lumen of the endoscope to the tissue, or a magnetic field concentrator may be passed through the lumen to the tissue.

FIG. 3 illustrates injection of magnetic nanoparticles into tissue to be treated.

FIG. 4 illustrates a magnetically permeable, flexible, rod passed through an endoscope to provide an AC magnetic field for heating magnetic nanoparticles in tissue to be treated.

FIG. 5 is a schematic diagram illustrating a simple homogeneous magnetically permeable rod adapted to fit within a lumen of an endoscope and to conduct magnetic fields originating in a coil.

FIG. 6 is a flowchart of a method of treating tissue using magnetic nanoparticles localized in the tissue, and an AC magnetic field applied through a magnetically permeable field concentrator.

FIG. 7 is a block-level diagram of a system adapted for planning and conducting magnetic nanoparticle treatment of selected tissues.

FIG. 8 is a plot of AC magnetic field strength produced by a flat coil.

FIG. 9 is a plot of AC magnetic field strength produced by the flat coil used in the plot of FIG. 8 with an added passive concentrator located 8 centimeters above coil center.

FIG. 10 illustrates differences between field strength maps produced by differing shapes of magnetic field concentrators.

FIG. 11 illustrates a C-shaped active concentrator configured for treatment of tissues near skin surface, the gap of the concentrator being placed near tissue to be treated.

FIG. 12 illustrates apparatus to enhance efficiency of battery charging of rechargeable implants in patients. The implants may include deep brain and other neurostimulators and other electronic devices.

### DETAILED DESCRIPTION OF THE EMBODIMENTS

In applying heat for thermotherapy to tissues using magnetic nanoparticles, it is believed that heating can be focused on selected target tissues, organs, or tumors by selectively applying nanoparticles to those tissues. Among our prior patent applications are applications directed towards trapping nanoparticles at selected tissues to better target nanoparticle heating. Similarly, it is believed that heating can be focused in target tissues if the AC magnetic field that activates and heats the nanoparticles is directed to be significantly more intense at the target tissues than in surface or other nearby tissues.

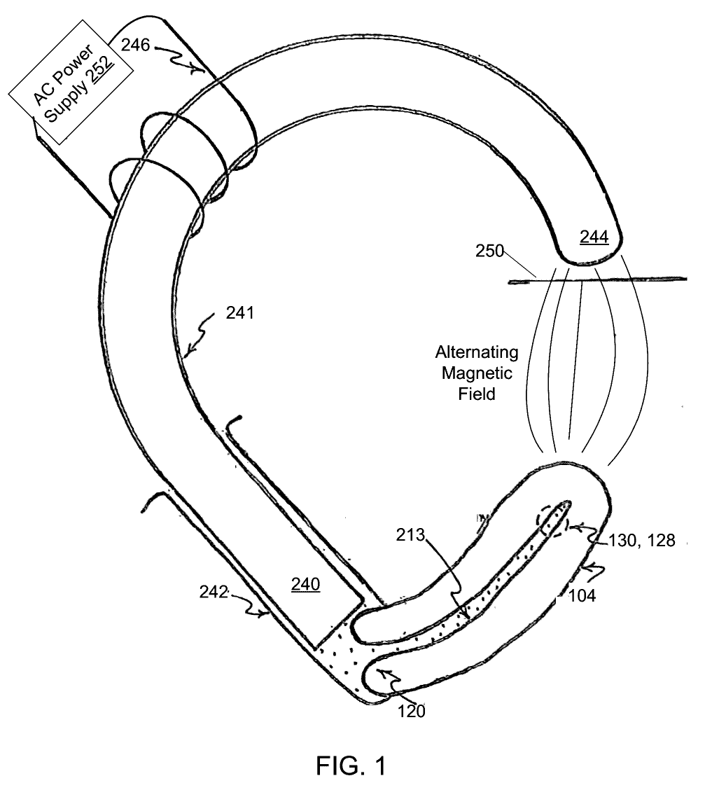

We have disclosed, in our application Ser. No. 13/945,610, using a flexible magnetically-permeable rod 241 (FIG. 1) having an end 240 inserted into the vagina, and a second end placed 242 placed over skin 250 over a fallopian tube. A magnetic heating composition, with in an embodiment is a magnetic nanoparticle suspension 130, is placed in the fallopian tube 128, and a magnetically permeable fluid 213 is injected into a lumen of the uterus 104. Apparatus, in an embodiment including a coil 246 wound over the flexible magnetically-permeable rod 241 and a high-frequency AC power supply 252 coupled to drive the coil, is configured for generating an AC magnetic field along the magnetically permeable rod. In the device of Ser. No. 13/940,610, the AC magnetic field is carried along the rod and magnetically-permeable liquid to provide an AC magnetic field particularly concentrated at the magnetic heating composition in the fallopian tube, thereby heating the fallopian tube without unduly heating body surface or nearby structures.

As illustrated in FIG. 1, a flexible magnetically-permeable rod has two ends. The first end 240 of the rod 241 is inserted 216 into the vagina 242. A second end 244 of the magnetically permeable rod is positioned over a first of the fallopian tubes to be heated. A high-frequency alternating current is then applied by an AC power supply 252 to energize 220 a coil 246 wound around the rod, causing a high-intensity, alternating, magnetic field to develop along the rod into the vagina, through the magnetically permeable fluid in the uterus, through the magnetic nanoparticle suspension in the fallopian tube, then through the abdominal wall 250 and abdominal tissue of the subject to the other end of the rod 244. The alternating magnetic field interacts with, and heats, the magnetic nanoparticle suspension in the fallopian tube In this way, the materials of high magnetic permeability form a path, albeit incomplete, through which the magnetic field induced by the coil will pass preferentially; this path includes the Fallopian tube and nanoparticle suspension, which is to be heated therapeutically by the magnetic material enclosed within it.

In a particular embodiment, the magnetically permeable fluid in the uterus is Ferridex, which has a high permeability (forms a preferred path for magnetic field lines), but it has no pronounced heating ability. By contrast, in this embodiment the fluid in the fallopian tubes is a suspension of iron oxide nanoparticles of about 65 nanometer core diameter, where each iron oxide nanoparticle has a biocompatible coating, and the iron oxide nanoparticles are optimized for high energy absorption from alternating magnetic fields.

In another embodiment, a flexible portion 304 of an endoscope, coupled to endoscope control head 306, is inserted through a bodily orifice, such as mouth 308, of a subject. The endoscope is equipped with an illuminator 305, guidance camera, and display monitor (not shown) as known in the art of medical endoscopes and colonoscopies such that insertion of the endoscope is performed by a surgeon operating control head 306 under visual control using images captured through a lens 352 (FIG. 3, 4) at endoscope tip 314, 350. The surgeon operates knobs 310 on control head 306 that are coupled through wires within flexible portion 304 to endoscope tip 314 so that the surgeon can guide the endoscope tip 314 through gut of the subject, such as through esophagus 312 and stomach 316 to position endoscope tip 314 near tissue to be treated.

In an alternative embodiment, the flexible portion 304 of the endoscope is inserted through an anus of the subject, and navigated through a colon of the subject, to place endoscope tip 314 near tissue to be treated. In yet another embodiment, the flexible portion of the endoscope is threaded through trachea and bronchi of the subject to place endoscope tip 314 near tissue to be treated.

Endoscope flexible portion 304 has a lumen running from control head 306 to endoscope tip 314, as is known in the art of medical endoscopes and often used for treatment of polyps, and of the type that is provided for use in procedures such as passing a catheter through the lumen and into the bile duct, pancreatic duct, or similar structures, or provided for passing a gripping tool through the lumen for recovery of swallowed objects.

Using an endoscope passed into a patient through the mouth, a surgeon can position the endoscope tip 314 adjacent to a tissue-to-be-treated location in the stomach 316, as well as in the upper duodenum 317. Among locations that can be reached with the endoscope tip are adjacent the Sphincter of Oddi, where the common bile duct opens into the duodenum 317; the common bile duct typically carries bile from the gallbladder (not shown) and liver 315 into the duodenum 317; similarly the major pancreatic duct from the pancreas 318 carries pancreatic enzymes into the bile duct at the Sphincter of Oddi. Objects, such as catheters, can be threaded through an endoscope through the sphincter into the bile duct or into the sphincter, and thence into liver or pancreas. Similarly, many patients have an accessory pancreatic duct that leads from part of the pancreas 318 directly into the duodenum 317, this may also be penetrated by objects threaded through the lumen of the endoscope. Endoscopes adapted to insertion through the anus (not shown) can similarly position endoscope tip 314 near many locations along the colon, including adjacent any diverticulae, polyps, or tumors that may exist, and entry to distal ileum is often possible, the opening of the appendix into the colon can also often be reached.

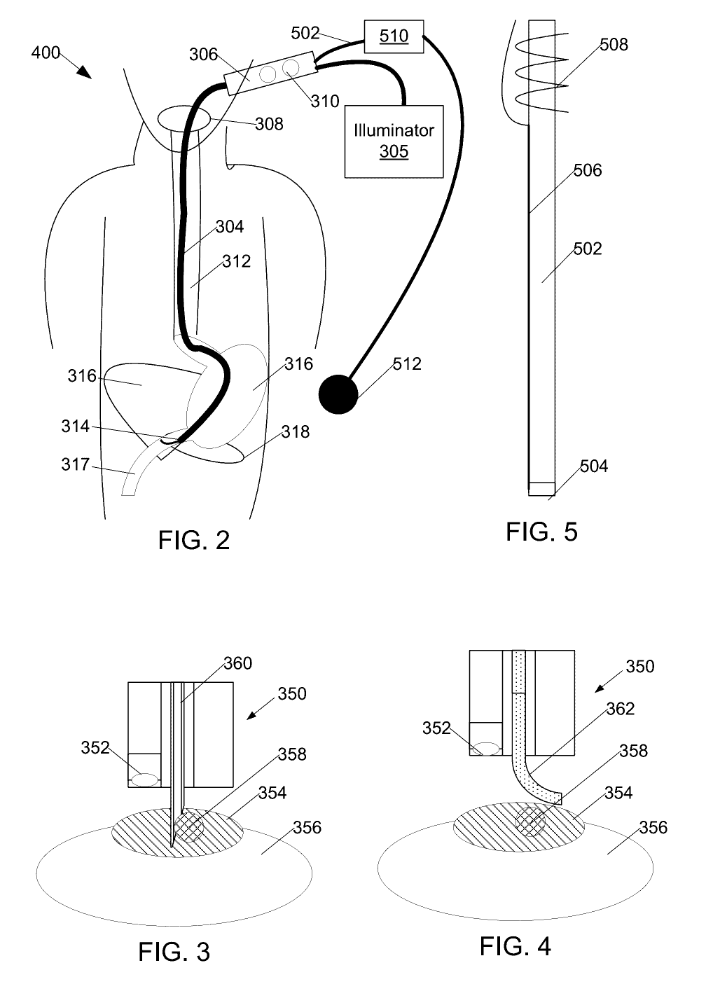

The magnetically permeable rod of FIG. 1 serves as a magnetic field concentrator and director, directing magnetic fields produced by coil 246 and providing a stronger AC magnetic field in certain areas of the body than would otherwise be available. Similarly, the endoscopic magnetically permeable rod of FIGS. 2, 3, 4, and 5 serves as a magnetic field concentrator and director, directing portions of the AC magnetic field to the tumor or other tissue to be treated. Since these embodiments have the coils wound around the concentrator, which serves as a core of the coil and raises inductance of the coil, we refer to them herein as active concentrators. We have found that the AC magnetic fields provided for magnetic nanoparticle thermotherapy may also be affected and directed by magnetically permeable shapes that do not extend into core of the coil, but are located at strategically-located places within the AC magnetic field; we call these passive concentrators.

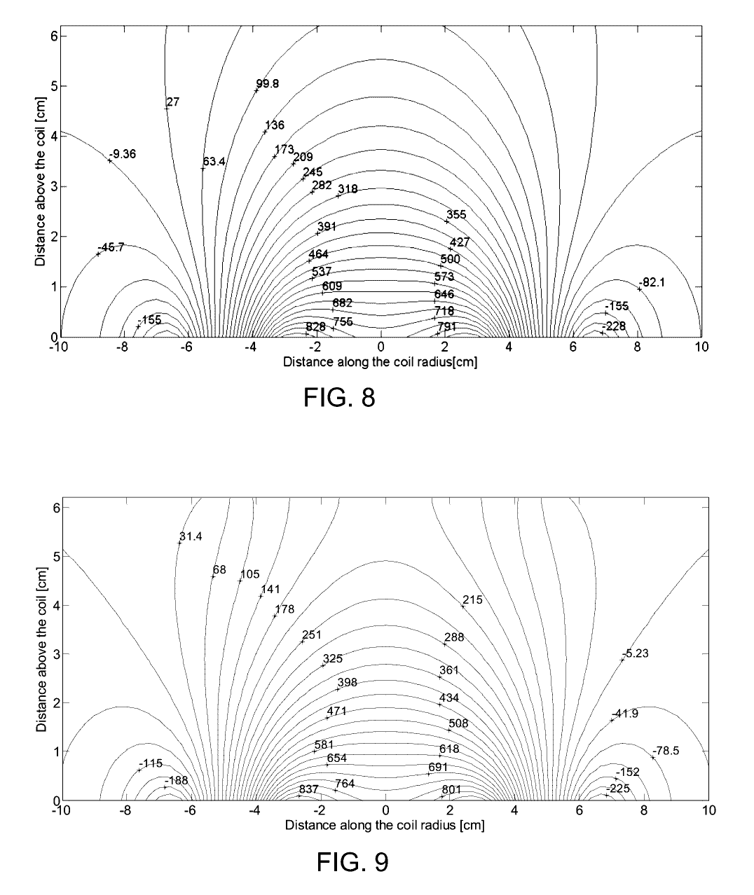

As an example of effect of a passive concentrator, FIG. 8 is a plot of field strength produced by a particular flat coil; while FIG. 9 is a plot of field strength produced by the flat coil of FIG. 8, with an added flat passive concentrator centered 8 centimeters over the coil. As can be seen, field strengths in the range of 3 to 6 centimeters above the coil is substantially stronger with the passive concentrator than without the concentrator. These dimensions can be scaled to dimensions suitable for use with patients, such that the volume of stronger field strength is located in a region of tissue to be treated.

Concentrators are formed of magnetically permeable materials. Some embodiments of passive and active concentrators are formed from ferrites similar to those used in the electronic industry as cores of inductors. Other embodiments are formed from finely divided particles of ferromagnetic materials, such as iron or iron oxide, embedded in a nonmagnetic polymer or ceramic, in a particular embodiment in a flexible silicone matrix. In some embodiments where the concentrator absorbs significant energy from applied AC magnetic fields, the concentrator is equipped with a cooling system such as a water-jacket for liquid cooling of the concentrator.

With reference to the endoscopic embodiment illustrated in FIGS. 2, 3, 4, and 5, the concentrator in some embodiments is an active concentrator that extends all the way from the tissue to be treated, through the lumen of the endoscope, and to the coil. In alternative embodiments, a larger coil external to the body is used and the concentrator is a passive concentrator. In an embodiment, the concentrator has a distal magnetically-permeable portion that extends only part of the distance from tissue to be treated and into the lumen of the endoscope, and a proximal nonmagnetic portion fabricated of a flexible nonmagnetic material. This embodiment with endoscope inserted through the mouth is expected to be useful in treating cancers of the such as, but not limited to, cancers of the esophagus, stomach, duodenum, bile duct, or pancreas, and when inserted through the rectum in treating prostate cancers and other abnormal tissues of the rectum and colon. In particular embodiments, the magnetic nanoparticles are injected directly through the gut wall through a needle and catheter inserted through the endoscope lumen into the tissue to be treated, the needle is withdrawn and concentrator positioned, then the external coil is energized to provide an AC magnetic field to heat the nanoparticles and treat the tissue.

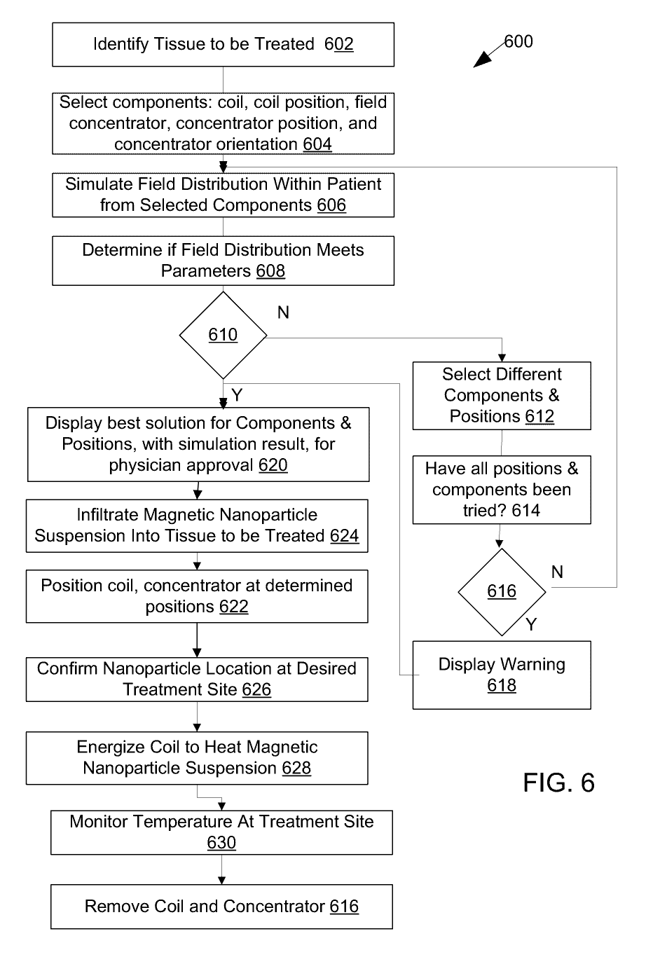

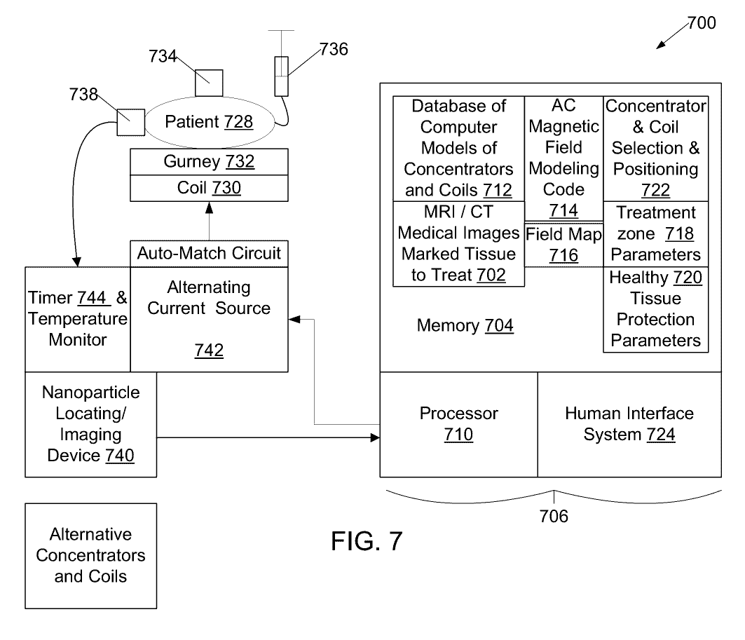

With reference to FIGS. 6 and 7, the method 600 begins with a surgeon, using medical images 702 obtained through X-Ray Computed Tomography (CT) or Magnetic Resonance Imaging (MRI), identifies 602, abnormal tissue requiring treatment, the abnormal tissue is typically adjacent to or near normal tissue that the surgeon does not wish to damage, the surgeon flags the tissue requiring treatment on the images 702 in memory 704. The medical images 702 are read into memory 704 of an image processing, magnetic simulation, display, and control computing system 706 of magnetic nanoparticle treatment system 700. The surgeon then selects 604 an initial selected coil with any active concentrator, passive magnetic field concentrator selection and position, and concentrator orientation. The surgeon also selects desired treatment zone parameters 718, including minimum field strengths at the tissue to be treated. Similarly, the surgeon selects healthy tissue protection parameters 720, including maximum field strengths to which non-treated tissue may be subjected to during treatment.

The processor 710 reads magnetic models of the selected coil and passive magnetic field concentrator from a library 712 of magnetic models of available components, and executes static field modeling code 714 to simulate 606 the magnetic fields and generate a map 716 of field strengths expected in the patient's body during treatment. The processor then compares field strength map 716 to treatment zone parameters 718 at the abnormal tissue position marked on medical images 702, and healthy tissue protection parameters 720 at other locations within a model of the patient, to determine 608 if parameters are met.

If parameters 718, 720 are not met by field map 716, concentrator and coil selection and positioning code 722 is executed to determine an alternative position; the simulation 606 and determining if parameters are met 608 are then repeated until all possible selections of available coil and concentrator components and positions are tested. If 616 all available selections have been tried with none meeting parameters, a warning is displayed 618 on human interface system 724.

The best simulated combination of coil selection, concentrator selection, coil position, patient position, and concentrator position and orientation, is displayed on human interface system 724 for physician approval 620; if approved the patient 728 is prepared for treatment on nonmagnetic gurney 732, and coil 730, and concentrator 734, are positioned at the determined positions 622. A magnetic nanoparticle preparation, such as a suspension 736, is administered by infiltrating the nanoparticles into the organ to be treated and, if any temperature sensor 738 is used during treatment, the sensor is positioned to observe temperature at the tissue to be treated. (we note that temperature sensors are preferably nonmagnetic) In embodiments, the nanoparticle suspension is injected into a bloodstream of the patient in such a way as to be taken up by the tissue to be treated, in some such embodiments it is injected through a catheter into an artery that feeds the tissue to be treated. In an alternative embodiment, the nanoparticle preparation is infiltrated through a catheter or needle directly into the tissue to be treated, the needle or catheter may or may not be inserted through an endoscope. Nanoparticle positions within the patient may in some embodiments be confirmed 626 with a magnetic nanoparticle detector or imaging system 740. Processor 710 then activates alternating current source 742, which passes current through a matching device into coil 730, thereby energizing 628 the coil to provide an AC magnetic field that is transported and shaped by active and passive concentrators to the nanoparticles, and thereby heats the nanoparticles in the tissue to be treated.

Treatment is continued until temperature at the treatment site, as monitored 630 through sensor 738, rises to a prescribed level or until a preset treatment timer 744 expires. In a particular embodiment where the patient is supported on a nonmagnetic gurney 732 or on a nonmagnetic tabletop such as a wood and/or plastic tabletop, the coil 730 is located beneath gurney 732 and, in order to prevent overheating of normal tissue while heating the nanoparticles in the tissue to be treated, the coil is moved from a first to a second position while energized to provide the AC magnetic field to heat the nanoparticles.

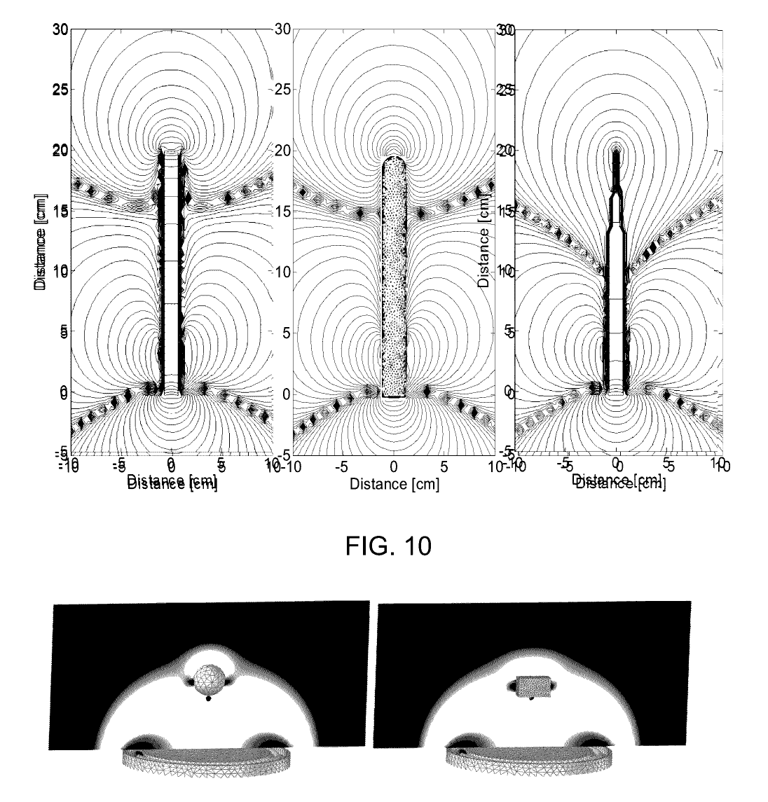

As an example but not by limitation, illustrated in FIG. 10 are three field strength maps, each produced by a different shape of concentrator.

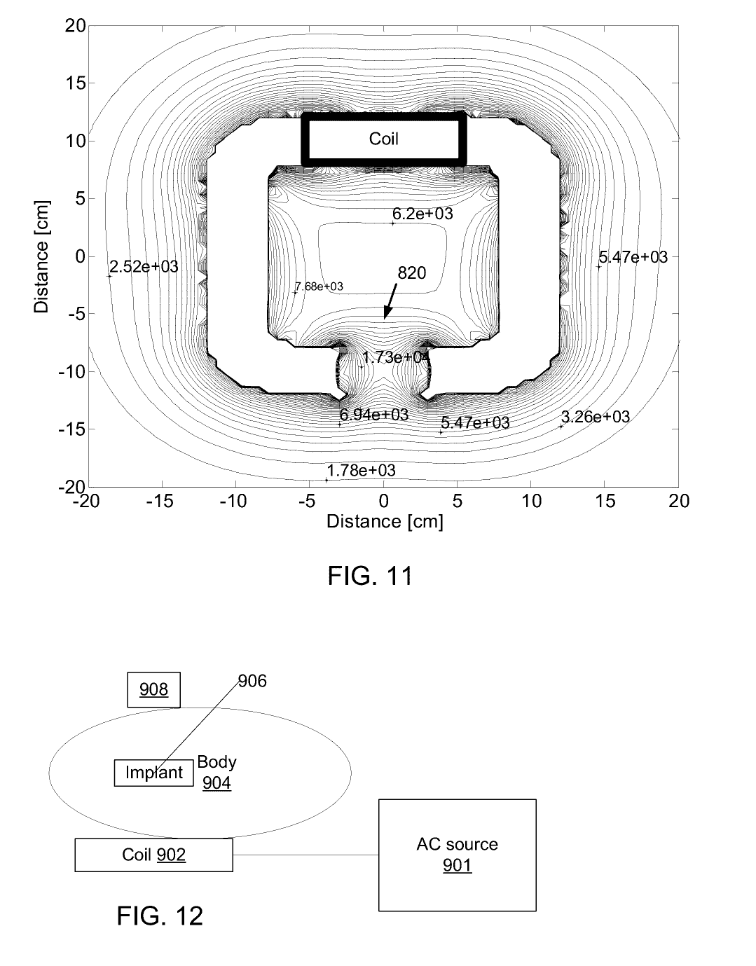

An embodiment adapted for treatment of abnormal tissues, including cancers, of the breast, head, or neck is illustrated in FIG. 11. This embodiment has a coil around the central portion of the U-shaped concentrator with a gap 820 between ends of the U-shaped concentrator arms. The gap is positioned around, or near, the tissue to be treated as intense fields are provided across the gap. For example, nanoparticles are infiltrated into abnormal tissues potentially including but not limited to a cancerous breast tumor or tumor in a jaw, the gap is positioned around the tumor, and AC current is applied to the coil to generate AC fields in the gap that heat the nanoparticles in the tumor.

Embodiments of the system herein described are adapted to treat cancerous tissue. In an embodiment, an active concentrator is adapted to being inserted into the rectum, while the coil associated with the active concentrator remains outside the body. This concentrator is used to treat either prostatic hypertrophy or prostate cancer by infiltrating nanoparticles into the prostate, inserting the concentrator into the rectum, and applying AC current to the coil to generate a magnetic field, the field conducted to the prostate, and thereby heating the prostate.

In an alternative embodiment, instead of injecting magnetic nanoparticles and applying an AC magnetic field to heat them, a passive concentrator is used to increase efficiency of battery charging of an implant as illustrated in FIG. 12. An AC source 901 is coupled to a coil 902 to provide an ac magnetic field. A patient is 904 having a rechargeable implant 906 is placed on coil 902. A concentrator 908 is placed on the patient to modify the AC magnetic field to optimize battery charging.

While the terms coil and AC current source have been used to describe apparatus for originating the AC magnetic field, we note that at VHF frequencies an AC current source and an antenna may serve to provide such fields. The term “source of AC magnetic fields” shall include both coil or antenna with an AC current source as a source of the AC magnetic field.

### Combinations

The various features herein described may be combined in various ways. Among the combinations of features anticipated by the inventors are:

A system designated A adapted for applying heat to a treatment location in a subject including an alternating current (AC) source coupled to energize a coil configured to generate an AC magnetic field; a magnetically permeable, passive magnetic concentrator formed of a magnetically permeable material, the concentrator not positioned within the coil, the concentrator positioned to alter an intensity distribution of the AC magnetic field; and apparatus configured to administer magnetic nanoparticles to tissue to be treated of a subject.

A system designated AA including the system designated A and further including a processor coupled to a memory, the memory containing a computer model of the coil and the concentrator, the memory further containing computer readable code for simulating magnetic fields and configured to generate an intensity map describing the intensity distribution of the AC magnetic field.

A system designated AB including the system designated A or AA the memory further containing treatment parameters and comparison code configured to compare simulated magnetic fields at the tissue to be treated to the treatment parameters.

A system designated AD including the system designated A, AA, or AB further including temperature monitoring apparatus configured to monitor temperature of the tissue to be treated during treatment.

A system designated AE including the system designated A, AA, or AB, or AD wherein the concentrator is provided with cooling apparatus.

A system designated AF including the system designated A, AA, AB, or AD or AE, and further including an endoscope, the apparatus configured to administer magnetic nanoparticles being configured with a needle adapted to being passed through a lumen of the endoscope.

A method designated B of providing an alternating current (AC) magnetic field to magnetic nanoparticles and heating the magnetic nanoparticles including providing a source of alternating current coupled to a coil; positioning the coil in a vicinity of the nanoparticles; positioning a passive concentrator near the nanoparticles; and energizing the coil with the alternating current to generate the AC magnetic field.

A method designated BA including the method designated B wherein the passive concentrator is adapted for insertion through an endoscope.

A method designated BB including the method designated B wherein the passive concentrator is adapted for insertion into a rectum.

A method designated BC including the method designated B and further including liquid cooling the concentrator.

A method designated BD including the method designated B, BA, BB, or BC and further including simulating the AC magnetic field and comparing the magnetic field to parameters to verify adequate heating of the nanoparticles.

A method designated BE including the method designated B, BA, BB, BC, or BD, and further including monitoring heating of the nanoparticles

While the invention has been particularly shown and described with reference to a preferred embodiment thereof, it will be understood by those skilled in the art that various other changes in the form and details may be made without departing from the spirit and scope of the invention. It is to be understood that various changes may be made in adapting the invention to different embodiments without departing from the broader inventive concepts disclosed herein and comprehended by the claims that follow.

What is claimed is:

1. A system adapted for applying heat to a treatment location in a subject comprising:
an alternating current (AC) source coupled to energize a coil configured to generate an AC magnetic field;
a magnetically permeable, passive magnetic concentrator formed of a magnetically permeable material, the concentrator not positioned within the coil, the concentrator positioned to alter an intensity distribution of the AC magnetic field; and
apparatus configured to administer magnetic nanoparticles to tissue to be treated of a subject.

2. A system adapted for applying heat to a treatment location in a subject comprising:
an alternating current (AC) source coupled to energize a coil configured to generate an AC magnetic field;
a magnetically permeable, passive magnetic concentrator formed of a magnetically permeable material, the concentrator not positioned within the coil, the concentrator positioned to alter an intensity distribution of the AC magnetic field;
apparatus configured to administer magnetic nanoparticles to tissue to be treated of a subject; and
a processor coupled to a memory, the memory containing a computer model of the coil and the concentrator, the memory further containing computer readable code for simulating magnetic fields and configured to generate an intensity map describing the intensity distribution of the AC magnetic field when executed by the processor.

3. The system of claim 2 the memory further containing treatment parameters and comparison code configured to compare simulated magnetic fields at the tissue to be treated to the treatment parameters.

4. The system of claim 3, further comprising temperature monitoring apparatus configured to monitor temperature of the tissue to be treated during treatment.

5. The system of claim 4 wherein the concentrator is provided with cooling apparatus.

6. The system of claim 4 further comprising an endoscope, the apparatus configured to administer magnetic nanoparticles being configured with a needle adapted to being passed through a lumen of the endoscope.

7. The system of claim 3 wherein the concentrator is provided with cooling apparatus.

8. The system of claim 3 further comprising an endoscope, the apparatus configured to administer magnetic nanoparticles being configured with a needle adapted to being passed through a lumen of the endoscope.

9. A method of providing an alternating current (AC) magnetic field to magnetic nanoparticles at a treatment location within a patient and heating the magnetic nanoparticles comprising:
providing a source of alternating current coupled to a coil;
positioning the coil to provide an AC magnetic field in a vicinity of the nanoparticles;
positioning a passive concentrator to adjust the AC magnetic field near the nanoparticles;
simulating the AC magnetic field to generate an intensity map describing the intensity distribution of the AC magnetic field and comparing the magnetic field to parameters to verify adequate heating of the nanoparticles while protecting healthy tissue of the patient; and
energizing the coil with the alternating current to generate the AC magnetic field, the AC magnetic field shaped by the passive concentrator and heat the magnetic nanoparticles.

10. The method of claim 9 wherein the passive concentrator is adapted for insertion through an endoscope.

11. The method of claim 9 wherein the passive concentrator is adapted for insertion into a rectum of the patient.

12. The method of claim 9 further comprising liquid cooling the concentrator.

13. The method of claim 9 and further comprising monitoring heating of the nanoparticles.

14. The method of claim 12 wherein the passive concentrator is adapted for insertion into a rectum.

15. The method of claim 14 and further comprising monitoring heating of the nanoparticles.
